(19) United States
(12) Patent Application Publication
(10) Pub. No.: US 2015/0128652 A1
(43) Pub. Date: May 14, 2015

(54) MESHWEAR WITH DIFFERENT ZONES IN STRESS-ELONGATION BEHAVIOUR

(71) Applicant: BAUERFEIND AG, Zeulenroda-Triebes (DE)

(72) Inventor: Denny Jung, Zeulenroda-Triebes (DE)

(73) Assignee: BAUERFEIND AG, Zeulenroda-Triebes (DE)

(21) Appl. No.: 14/381,845

(22) PCT Filed: Feb. 27, 2013
(86) PCT No.: PCT/EP2013/053958

§ 371 Date: Aug. 28, 2014

(30) Foreign Application Priority Data
Feb. 28, 2012 (DE) 10 2012 004 150.0

(51) Int. Cl. D04B 1/18; A61F 13/00; A61F 13/08

(52) U.S. Cl. CPC D04B 1/18; A61F 13/08; A61F 13/00038
USPC 66/178 A; 66/202; 28/142; 28/155; 602/53

(57) ABSTRACT

The invention relates to knit fabric having zones having normal stress-elongation behavior and having predefined zones having increased stress-elongation behavior, for example, knitted fabrics of this type, in particular knit fabric of this type used as medical aids or sport aids, and methods for producing this knit fabric.

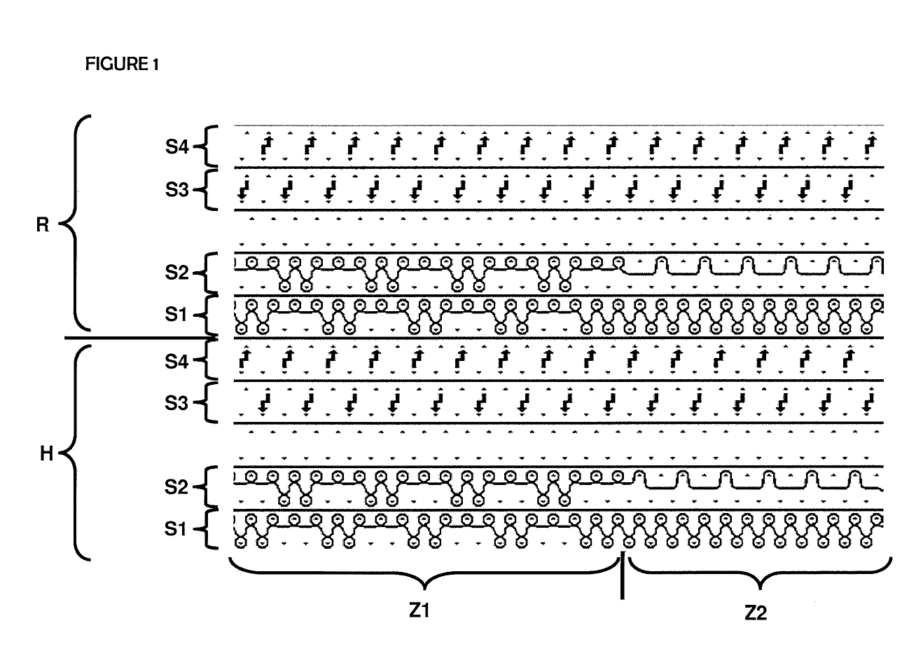

The invention relates to knit fabric having zones having normal stress-elongation behavior and having predefined zones having increased stress-elongation behavior, for example, knitted fabrics of this type, in particular knit fabric of this type used as medical aids or sport aids, and methods for producing this knit fabric.

Knit fabric, in particular knitted fabrics, having zones having normal stress-elongation behavior and having predefined zones having increased stress-elongation behavior, is known. Compression sleeves and stockings, which are knitted on a right-left circular knitting machine, having regions of lesser elasticity are known, for example, from DE 199 46 019 A1. In this case, the lesser elasticity is achieved by preventing the loop formation or by gluing or welding on a film material, for example, a plastic web. DE 20 2004 003 417 U1 describes knitted fabrics having regions of differing extensibility, wherein the extensibility differences are produced by differing incorporation of weft threads.

The invention has the object of providing knit fabric having zones having normal stress-elongation behavior and having predefined zones having increased stress-elongation behavior, for example, knitted fabrics of this type, wherein these zones having normal stress-elongation behavior and these zones having increased stress-elongation behavior are producible in an improved and simplified manner. The invention additionally has the object of providing improved and simplified methods for producing knit fabric having zones having normal stress-elongation behavior and having predefined zones having increased stress-elongation behavior, for example, of knitted fabrics of this type.

The technical problem on which the invention is based is solved by providing a knitted fabric and methods according to the main claim and the other independent claims and in particular by knit fabric having zones having normal stress-elongation behavior and having predefined zones having increased stress-elongation behavior, wherein at least one loop row of the knit fabric has both at least one zone having normal stress-elongation behavior and also at least one zone having increased stress-elongation behavior, and wherein the knit fabric is stretchable both in the transverse direction and also in the longitudinal direction.

In a preferred embodiment, the knit fabric is a knitted fabric. In a preferred embodiment, the knit fabric is a flat knitted fabric. In a preferred embodiment, the knit fabric is three-dimensional. In a preferred embodiment, the knit fabric is a three-dimensional flat knitted fabric, for example, a bandage.

In conjunction with the present invention, a knitted fabric is understood as knit fabric produced by knitting, i.e., in particular not a net fabric or woven fabric. The preferred knitted fabric according to the invention can be in particular a compression knitted fabric.

In a preferred embodiment, a base thread is largely formed as loops in the zones having normal stress-elongation behavior, and the base thread is at least partially formed as shortened loops, tuck stitches, and/or floats in the zones having increased stress-elongation behavior, so that the length of the base thread per longitudinal section of the loop row in the zones having increased stress-elongation behavior is shorter than the length of the base thread per longitudinal section of the loop row in the zones having normal stress-elongation behavior.

In a preferred embodiment, a base thread is largely formed as loops in the zones having normal stress-elongation behavior, and the base thread is at least partially formed as a tuck stitch and/or float in the zones having increased stress-elongation behavior, so that the length of the base thread per longitudinal section of the loop row in the zones having increased stress-elongation behavior is shorter than the length of the base thread or longitudinal section of the loop row in the zones having normal stress-elongation behavior.

The inventors of the present invention have found that in individual loop rows of knit fabric, in particular of a flat knitted fabric, both zones having normal stress-elongation behavior and also zones having increased stress-elongation behavior can be introduced in a simple manner. This can be achieved in particular in that a zone having increased stress-elongation behavior in the loop row has a higher number of tuck stitches and/or floats than a zone having normal stress-elongation behavior. By increasing the number of tuck stitches and/or floats, the length of the base thread in the zones having increased stress-elongation behavior per defined longitudinal section of a loop row, for example, 1 cm, can be shortened in comparison to the length of the base thread in the zones having normal stress-elongation behavior or equal longitudinal section of the loop row, i.e., for example, also 1 cm. The shortening of the thread length per unit of length of the loop row represents a shortened thread profile and results in reduced elasticity and therefore an increased stress-elongation behavior.

Such knit fabric is advantageously stretchable both in the transverse direction and also in the longitudinal direction.

A loop row can be formed, for example, from loops, tuck stitches, and/or floats. A loop row is preferably primarily formed from loops, between which in specific regions or zones, in particular zones having increased stress-elongation behavior, predominantly tuck stitches and/or floats are formed.

Alternatively or additionally to the use of tuck stitches and/or floats, shortening of the loops can also be used to increase the stress-elongation behavior in a zone.

The zones having normal stress-elongation behavior and the zones having increased stress-elongation behavior are formed by corresponding sections of at least one loop row, in particular a plurality of loop rows.

The elasticity, i.e., the stress-elongation behavior, can advantageously be set by the number of tuck stitches and/or floats in a zone, i.e., a corresponding section of at least one loop row. A high number of tuck stitches and/or floats in a zone, or in a section of a loop row, results in a low elasticity and an increased stress-elongation behavior in this zone or in this section of the loop row, respectively.

In this case, a specific number of floats in a zone results in a lower elasticity and a higher stress-elongation behavior than the same number of tuck stitches in a zone of equal size.

Therefore, zones having normal stress-elongation behavior can also have tuck stitches and/or floats, as long as the zones having increased stress-elongation behavior have more tuck stitches and/or floats or have such a loop/tuck stitch/float ratio, which results in a higher stress-elongation behavior.

In a preferred embodiment, the zones having normal stress-elongation behavior have more loops per longitudinal section of the loop row than zones having increased stress-elongation behavior in a longitudinal section of equal length of the mesh row.

In a preferred embodiment, the zones having normal stress-elongation behavior have fewer tuck stitches and/or floats per longitudinal section of the loop row than zones having increased stress-elongation behavior in a longitudinal section of equal length of the loop row.

The loops, tuck stitches, and floats of a loop row and therefore the knit fabric are formed in particular by the base thread. Of course, the loops, tuck stitches, and floats can also be formed from further threads, however, wherein the further threads can be provided in addition to the base thread, for example, as plating threads, or are provided alternatively to the base thread, for example, as further, different-colored or different-natured second base threads for a different-colored or different-natured region of the knit fabric.

In a preferred embodiment, the knit fabric has a base thread and at least one further thread.

Knit fabric according to the invention contains at least one loop row, in particular a plurality of loop rows.

In the case of preferred three-dimensional knit fabric, a loop row forms a peripheral plane of the three-dimensional knit fabric.

In a preferred embodiment, the knit fabric is a correctly-shaped, in particular three-dimensional correctly-shaped knit fabric. “Correctly-shaped” is understood in particular to mean that the knit fabric adapts itself or is adapted in its profile to a body shape.

According to the invention, the zones having increased stress-elongation behavior are predefined zones having increased stress-elongation behavior. They are specific and defined zones, which have an increased stress-elongation behavior, to thus be able to exert a desired compression effect on specific body regions, for example, in the case of a bandage. In particular by way of the production method according to the invention, the zones having increased stress-elongation behavior can be predefined easily and in an advantageous manner during the production of the knit fabric by predefining the positioning a number of tuck stitches and/or floats.

In a preferred embodiment, the knit fabric is based on a right/right weave and/or an at least 1-bed weave. In a preferred embodiment, the knit fabric is based on a right/right weave and/or an at least 2-bed weave. In a preferred embodiment, the knit fabric is based on a right/right weave and/or a 2-bed, 3-bed, or 4-bed weave. In a preferred embodiment, the knit fabric is based on a right/right weave and/or a 2-bed weave. In a preferred embodiment, the knit fabric is based on a right/right weave. In an alternative embodiment, the knit fabric is based on a left/left weave.

The invention also solves the technical problem on which it is based by way of a method for producing knit fabric having zones having normal stress-elongation behavior and having predefined zones having increased loop-elongation behavior from a base thread and at least one further thread on a loop-forming machine having loop-forming elements in at least two planes, wherein the zones having normal stress behavior are produced by way of the at least partial formation of the base thread as loops, and the zones having increased stress-elongation behavior are produced by way of the partial formation of the base thread as shortened loops, as tuck stitches, and/or as floats on at least one of the at least two planes having loop-forming elements.

A method is preferred for producing knit fabric having zones having normal stress-elongation behavior and having predefined zones having increased stress-elongation behavior from a base thread and at least one further thread on a loop-forming machine having loop-forming elements in at least two planes, wherein the zones having normal stress behavior are produced by way of the at least partial formation of the base thread as loops, and the zones having increased stress-elongation behavior are produced by way of the partial formation of the base thread as tuck stitches and/or as floats on at least one of the at least two planes having loop-forming elements.

In a preferred embodiment, the zones having increased stress-elongation behavior are produced by way of the partial formation of the base thread as shortened loops, as tuck stitches, and/or as a float on one of the at least two planes having loop-forming elements. In an alternative embodiment, the zones having increased stress-elongation behavior are produced by way of the partial formation of the base thread as shortened loops, as tuck stitches, and/or as floats on two of the at least two planes having loop-forming elements. In a preferred embodiment, the zones having increased stress-elongation behavior are produced by the partial formation of the base thread as shortened loops, as tuck stitches, and/or as floats on at least two of the at least two planes having loop-forming elements.

In a preferred embodiment, the loop-forming machine having loop-forming elements in at least two planes is a knitting machine, in particular a flat knitting machine.

In a preferred embodiment, a plating thread is associated with the base thread in at least one loop and/or at least one tuck stitch and/or at least one float.

In a preferred embodiment, no inelastic weft thread is used in the method according to the invention. In a preferred embodiment, an inelastic weft thread is not introduced, for example, inserted, into the knit fabric in the method according to the invention.

In a preferred embodiment, by way of further systems and/or devices of the loop-forming machine, a translocalization of the base thread and/or the plating thread and/or the at least one further thread takes place between the at least two planes having loop-forming elements of the loop-forming machine.

A translocalization is preferably understood as rehanging of the base thread and/or the plating thread and/or the at least one further thread.

In a preferred embodiment, at least one elastic weft thread is inserted and/or incorporated in the knit fabric.

In a preferred embodiment, the method according to the invention is used for producing a knitted fabric, in particular a flat knitted fabric.

In a preferred embodiment, the method according to the invention is used for producing knit fabric according to the invention.

The invention also solves the technical problem on which it is based by way of a method for producing knit fabric, in particular compression knit fabric, from a base thread and at least one further thread on a loop-forming machine having loop-forming elements in at least two planes, wherein the base thread and/or the at least one further thread are at least partially translocalized between the at least two planes.

In a preferred embodiment, knit fabric thus produced is knit fabric according to the invention, in particular compression knit fabric according to the invention.

In a preferred embodiment of the method, at least one loop row of the knit fabric has both at least one zone having normal stress-elongation behavior and also at least one zone having increased stress-elongation behavior.

The inventors of the present invention have surprisingly found that an at least partial translocalization, in particular an at least partial rehanging of the base thread and/or the at least one further thread at least partially between the at least two planes of a loop-forming element having loop-forming elements in at least two planes, a particularly breathable knit fabric can be obtained, which is more pleasant to wear, in particular compression knit fabric, for example, a bandage. The rehanging results in a network pattern in the knit fabric which was heretofore unknown in bandages.

In a preferred embodiment, the compression knit fabric obtainable by the above-mentioned method is knit fabric having zones having normal stress-elongation behavior and having predefined zones having increased stress-elongation behavior, in particular knit fabric according to the invention.

The present invention also relates to knit fabric, in particular knit fabric described in this text, producible by a method according to the invention, in particular produced by a method according to the invention.

In a preferred embodiment, the at least one loop row of the knit fabric has both at least one zone having normal stress-elongation behavior and also at least one zone having increased stress-elongation behavior, wherein the knit fabric is stretchable both in the transverse direction and also in the longitudinal direction.

In a preferred embodiment, knit fabric according to the invention does not have an inelastic weft thread, in particular does not have an inelastic weft thread which is to cause or causes an increased stress-elongation behavior in specific zones of the knit fabric. The use of inelastic weft threads, which are to cause an increased stress-elongation behavior in specific zones of the knit fabric, as are used in the prior art, results in a strong reduction or prevention of the extensibility either in the longitudinal direction or in the transverse direction.

The present invention also relates to medical aids or sport aids, containing or consisting of knit fabric according to the invention.

The present invention also relates to a bandage, containing or consisting of knit fabric according to the invention.

The bandage according to the invention can be, for example, a medical bandage or a sports bandage.

In a preferred embodiment, the bandage is a compression bandage.

The present invention also relates to a compression stocking, containing or consisting of knit fabric according to the invention.

It has been shown that the knit fabric according to the invention may be used as compression bandages or compression stockings, wherein the zones produced according to the invention having increased stress-elongation behavior result in sufficient compression for at least compression class III.

Compression classes I to IV are defined on the basis of RAL GZ (RAL Quality Certification Mark) 387/1 (quality and testing definition for medical compression bandages) January 2008 edition, page 14.

It is preferable in this case for the at least one zone having increased stress-elongation behavior to correspond to compression class III or IV, i.e., to exert a compression of at least 4.5 kPa. The at least one zone having increased stress-elongation behavior preferably corresponds to compression class III, i.e., it exerts a compression of at least 4.5 kPa and at most 6.1 kPa. Alternatively, the at least one zone having increased stress-elongation behavior corresponds to compression class IV, i.e., it exerts a compression of at least 6.5 kPa.

It is also preferable for the at least one zone having normal stress-elongation behavior to correspond to compression class I or II, i.e., to exert a compression of at most 4.3 kPa. The at least one zone having normal stress-elongation behavior preferably corresponds to compression class II, i.e., it exerts a compression of at least 3.1 kPa and at most 4.3 kPa. Alternatively, the at least one zone having normal stress-elongation behavior corresponds to compression class I, i.e., it exerts a compression of at most 2.8 kPa.

The at least one zone having increased stress-elongation behavior preferably corresponds to compression class III, i.e., it exerts a compression of at least 4.5 kPa and at most 6.1 kPa and the at least one zone having normal stress-elongation behavior corresponds to compression class II, i.e., it exerts a compression of at least 3.1 kPa and at most 4.3 kPa.

In a preferred embodiment, the at least one zone having increased stress-elongation behavior of the bandage exerts a compression of at least 4.5 kPa.

It has also surprisingly been shown that the zones produced according to the invention having increased stress-elongation behavior can reinforce or replace the effect produced by a support pad.

In one embodiment, a medical aid according to the invention, for example, a bandage according to the invention or a compression stocking according to the invention, therefore has a support pad. In a preferred embodiment, the at least one zone having increased stress-elongation behavior of the knit fabric can be associated with the support pad in this case. For example, the at least one zone having increased stress-elongation behavior can cover the support pad. In this case, the support pad is preferably connected to the at least one zone having increased stress-elongation behavior, for example, sewn or glued or welded on.

In an alternative embodiment, a medical aid according to the invention, for example, a bandage according to the invention or a compression stocking according to the invention, does not have a support pad. In a preferred embodiment, the at least one zone having increased stress-elongation behavior of the knit fabric is positioned in this case so that it replaces a support pad.

The present invention also relates to a medical aid or a sport aid, containing knit fabric according to the invention, in particular a knitted fabric according to the invention or consisting of knit fabric according to the invention, in particular a knitted fabric according to the invention.

In a preferred embodiment, the bandage does not have any additional stabilizing elements, guide elements, and/or positioning aids.

The inventors of the present invention have surprisingly found that in the bandages made of knit fabric according to the invention, such a strong compression and/or stabilization can be produced by the zones having increased stress-elongation behavior that additional stabilizing elements, guide elements, and/or positioning aids can be omitted if needed. A bandage can thus be produced more simply and cost-effectively, wherein increased wearing comfort and a weight reduction are achieved simultaneously. In addition, knit fabric according to the invention in the form of a bandage is surprisingly so slip proof that no further aids are needed to prevent slipping.

The present invention thus also relates to the use of knit fabric according to the invention, in particular knitted fabric according to the invention as medical aids or as sport aids, in particular a bandage or compression stocking, wherein the at least one zone having increased stress-elongation behavior is used for preventing slipping of the bandage.

The present invention thus also relates to the use of knit fabric according to the invention, in particular knitted fabric according to the invention, as medical aids or as sport aids, in particular a bandage or compression stocking, wherein the at least one zone having increased stress-elongation behavior is used as a positioning aid for positioning the bandage.

Of course, additional stabilizing elements, guide elements, and/or positioning aids can also be provided, for example, in special medical embodiments of the bandage.

Of course, a bandage according to the invention can also have additional pressure-minimized regions, in particular on the top and bottom regions of the bandage. These pressure-minimized regions do not have to consist of knit fabric according to the invention.

The present invention also relates to the use of knit fabric according to the invention, in particular knitted fabric according to the invention, as medical aids or sport aids.

The present invention preferably also relates to the use of knit fabric according to the invention, in particular knitted fabric according to the invention, as medical aids. In particular in the case of medical aids, the zones having increased stress-elongation behavior are to support the adjoining body region.

The present invention also relates to the use of knit fabric according to the invention, in particular knitted fabric according to the invention, in particular as a compression bandage.

The present invention also relates to the use of knit fabric according to the invention as a compression stocking.

A use is preferred, wherein the at least one zone having increased stress-elongation behavior supports the adjoining body region and exerts a compression of at least 4.5 kPa, i.e., corresponds to compression class III or IV, in particular III. A use is also preferred, wherein the at least one zone having normal stress-elongation behavior supports the adjoining body region and exerts a compression of at most 4.3 kPa, i.e., corresponds to compression class II or I, in particular II.

The present invention thus also relates to the use of knit fabric according to the invention, in particular knitted fabric according to the invention as sport aids. In particular in sport aids, the stiffening achieved according to the invention can be used for taping effects. The knit fabric can also be used for a fatigue-inhibiting interaction with underlying muscle tissue.

The present invention thus also relates to the use of knit fabric according to the invention, in particular knitted fabric according to the invention, as medical aids or in particular as sport aids, in particular a bandage or compression stocking, wherein the at least one zone having increased stress-elongation behavior is used as a taping zone or as a fatigue-inhibiting zone.

The uses according to the invention also include corresponding embodiment forms of the products according to the invention, in particular knit fabric, knitted fabrics, medical products, sport products, bandages, and compression stockings, as being included with the invention.

Preferred embodiments result from the dependent claims.

The invention will be explained in greater detail hereafter on the basis of the exemplary embodiment shown in the drawing, without a restriction of the invention taking place.

In this case, the FIGURE shows: A schematic thread profile during a knitting method according to the invention on a flat knitting machine having four systems and two needle beds.

### EXAMPLE

The FIGURE shows the schematic thread profile for the forward stroke (H) and the reverse stroke (R) during a preferred method according to the invention for producing a knitted fabric according to the invention, in which four knitting systems (S1, S2, S3, S4) are used to produce a loop row.

A thread system made of base thread with plating thread and a further thread is formed largely as loops in the systems S1 and S2 in the knitting zone having normal stress-elongation behavior (Z1) on both needle beds, while the thread system is formed in the knitting zone as loops having increased stress-elongation behavior (Z2) in the system S1 on both needle beds and as a tuck stitches and floats in system S2 on only one of the two needle beds. This results in lesser extensibility of this knitted zone having increased stress-elongation behavior due to the thread length, which is reduced by knitting technology, per unit of length and therefore also in increased stress-elongation behavior.

In the systems S3 and S4, the thread system is regularly rehung both in the knitting zone having normal stress-elongation behavior (Z1) and also in the knitting zone having increased stress-elongation behavior (Z2). This results in surprisingly breathable knitted fabric, wherein the fabric is nonetheless sufficiently reduced in elasticity in particular in the knitting zone having increased stress-elongation behavior (Z2).

1. A knit fabric having zones having normal stress-elongation behavior and having predefined zones having increased stress-elongation behavior, wherein at least one mesh row of the knit fabric has both at least one zone having normal stress-elongation behavior and also at least one zone having increased stress-elongation behavior, wherein in the zones having normal stress-elongation behavior, a base thread is largely formed as loops and in the zones having increased stress-elongation behavior, the base thread is at least partially formed as tuck stitches and/or floats, so that a first length of the base thread per longitudinal section of the loop row in the zones having increased stress-elongation behavior is shorter than a second length of the base thread per longitudinal section of the loop row in the zones having normal stress-elongation behavior, and wherein the knit fabric is stretchable both in a transverse direction and also in a longitudinal direction.

2-29. (canceled)

30. The knit fabric according to claim 1, wherein the knit fabric is a flat knitted fabric.

31. The knit fabric according to claim 1, wherein the knit fabric is three-dimensional.

32. The knit fabric according to claim 1, wherein the knit fabric is based on a right-right weave and/or an at least 1-bed weave.

33. A method for producing knit fabric having zones having normal stress-elongation behavior and having predefined zones having increased stress-elongation behavior made of a base thread and at least one further thread on a loop-forming machine having loop-forming elements in at least two planes, wherein the zones having normal stress-elongation behavior are produced by the at least partial implementation of the base thread as loops and the zones having increased stress-elongation behavior are produced by the partial implementation of the base thread as tuck stitches and/or floats on at least one of the at least two planes having loop-forming elements.

34. The method according to claim 33, wherein at least one loop row of the knit fabric has both at least one zone having normal stress-elongation behavior and also at least one zone having increased stress-elongation behavior.

35. The method according to claim 33, wherein a plating thread in at least one loop and/or at least one tuck stitch and/or at least one float is associated with the base thread.

36. The method according to claim 33, wherein a translocalization of the base thread and/or the plating thread and/or the at least one further thread between the at least two planes having loop-forming elements of the loop-forming machine takes place by way of further systems and/or devices of the loop-forming machine.

37. The method according to claim 33, wherein at least one elastic weft thread is inserted and/or incorporated into the knit fabric.

38. The knit fabric according to claim 1, wherein at least one loop row of the knit fabric has both at least one zone having normal stress-elongation behavior and also at least one zone having increased stress-elongation behavior, and wherein the knit fabric is extensible both in the transverse direction and also in the longitudinal direction.

39. The knit fabric according to claim 1, wherein the knit fabric does not have an inelastic weft thread.

40. The knit fabric of claim 1 in combination with a medical aid or a sport aid.

41. The knit fabric of claim 1 in combination with a bandage.

42. The bandage according to claim 41 wherein the bandage is a compression bandage.

43. The knit fabric of claim 1 in combination with a compression stocking.

44. The bandage according to claim 41, wherein the at least one zone having increased stress-elongation behavior of the knit fabric exerts a compression of at least 4.5 kPa.

45. The bandage according to claim 41, further comprising a support pad, wherein the at least one zone having increased stress-elongation behavior of the knit fabric is associated with the support pad.

46. The bandage according to claim 42, wherein the bandage does not have any additional stabilizing elements, guide elements, and/or positioning aids.

47. The compression stocking according to claim 43, wherein the at least one zone having increased stress-elongation behavior of the knit fabric exerts a compression of at least 4.5 kPa.

48. The compression stocking according to claim 43, further comprising a support pad, wherein the at least one zone having increased stress-elongation behavior of the knit fabric is associated with the support pad.
